(19) United States
(12) Patent Application Publication
(10) Pub. No.: US 2006/0293760 A1
(43) Pub. Date: Dec. 28, 2006

(54) Soft tissue implants with improved interfaces

(76) Inventor: Patrick G. DeDeyne, Duxbury, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/166,793

(22) Filed: Jun. 24, 2005

(51) Int. Cl. A61F 2/02

(52) U.S. Cl. 623/23.76

(57) ABSTRACT

Specialization of the end(s) or host tissue contact points of biocompatible scaffolds brings a functionality to the scaffold that facilitates the dual function of inducing new tissue formation and facilitating attachment and site-specific tissue formation at the scaffolds' functionalized points of fixation.

### BACKGROUND OF THE INVENTION

1. Field of the Invention

This invention is concerned with improving the interface of soft tissue implants with host tissue.

2. Related Art

Most approaches in tissue engineering employ scaffolds that focus on remodeling/regenerating a single tissue type. However, in most clinical situations the scaffold will bridge several tissue types or at least form the interface between two tissues. For instance, a scaffold designed to replace the anterior cruciate ligament will require bony in growth at the tips while fostering the formation of dense regular connective tissue the center, a scaffold that is designed to regenerate tendon needs to interface with skeletal muscle at one end whereas the other end interfaces with bone, a scaffold that may regenerate a spinal ligament or even the intervertebral disc needs not only to facilitate the regeneration of the pathological tissue but also foster a firm attachment of the scaffold to the vertebrae, where integration of the scaffold with the host tissue is imperative.

Several patents (Badylak U.S. Pat. No. 6,485,723, Voytik-Harbin U.S. Pat. No. 6,444,229, Voytik Harbin U.S. Pat. No. 6,264,992, the disclosures of which are hereby incorporated by reference) describe the use of biocompatible and bioresorbable extracellular matrix (ECM) materials, such as small intestine submucosa (SIS), in soft tissue remodeling and other patents and patent applications describe its use as a hybrid between SIS and synthetic material (Plouhar U.S. Pat. No. 6,638,312; Brown US 2003/0023316; Malaviya US2003/0021827; Plouhar US2002/0038151) or as a porous material (Malaviya US 2003/0049299 the disclosures of which are hereby incorporated by reference). However, little attention is paid to the specific requirements that a scaffold would require for a clinical application in which the SIS scaffold is attached to the bone interface (e.g., tendon insertion, ligament attachment). On the other hand, non-SIS-based collagen scaffolds can be utilized to facilitate bone in growth (i.e., Healos® Bone Graft available from DePuy Spine, Inc.) and is discussed by Silver U.S. Pat. No. 4,970,298; Constanz U.S. Pat. No. 5,231,169; Constanz U.S. Pat. No. 5,455,231; Silver U.S. Pat. No. 5,739,286; Silver U.S. Pat. No. 5,532,217; Kwan U.S. Pat. No. 5,776,193; Kwan U.S. Pat. No. 6,187,047, the disclosures of which are hereby incorporated by reference. Despite overwhelming evidence that SIS-based scaffolds can drive soft tissue remodeling in a number of tissues it is well recognized that SIS has limited potential for bone formation, unless it can be modified.

Therefore, there is a need to provide soft tissue implants that perform its intended soft tissue function while having improved ability to attach to other tissue. One such advance in soft tissue implants is provided for by the invention hereinafter disclosed.

### BRIEF DESCRIPTION OF THE DRAWINGS

FIG. 1 depicts a soft tissue implant with a portion of the implant's ends treated to encourage implant to tissue attachment.

FIG. 2 depicts a soft tissue implant with a portion of one of the implant's ends treated to encourage implant to tissue attachment.

FIG. 3 represents an intervertebral disc implant with vertebra contacting ends treated to encourage implant to vertebra attachment.

FIG. 4 shows cartilage implants with bone contacting surfaces treated to encourage implant to bone attachment.

### SUMMARY OF THE INVENTION

By specializing the end or an attachment point of a scaffold we bring a functionality to the scaffold that facilitates a dual function. On one hand it will induce new tissue formation and on the other hand it facilitate adhesion and site-specific tissue formation at its point of fixation. This approach will facilitate and accelerate the integration of the scaffold with the surrounding host tissue.

The purpose of the invention is to facilitate tissue specific regeneration in an orthopaedic application. More specifically, we disclose a method, which can create a scaffold simultaneously facilitates the regeneration of tissue between tendon/bone, ligament/bone, and ligament/muscle at the periphery of the scaffold interface (or other desired host tissue/implant contact point(s)) and generates vascular fibrous tissue or connective tissue in the center of or along the scaffold.

Thus, one aspect of this invention relates to an implant for attaching to host tissue comprising a biocompatible scaffold having at least one end or one point for contacting the host tissue, wherein the end or point of contact further comprises a tissue interface composition that permits host tissue ingrowth between the implant and the host tissue.

In one preferred embodiment, this invention is based on modifying an SIS scaffold so that it can facilitate bone in growth at its attachment points and soft tissue remodeling in the center of the scaffold. At the periphery the SIS scaffold is the collagen that may be mineralized and would allow bone ingrowth.

Another aspect of this invention relates to method of implanting a medical device comprising the steps of:

a) providing a scaffold having at least a first end or first contact point and a second end wherein the first end or contact point further comprises a tissue interface composition that permits tissue ingrowth between the device and the host tissue; and

b) contacting the end or contact point comprising the tissue interface composition with the host tissue.

### DETAILED DESCRIPTION OF PREFERRED EMBODIMENTS OF THE INVENTION

While most tissue engineering initiatives and strategies focus on methods and materials that can regenerate or remodel a single tissue (e.g., bone, cartilage, nucleus pulposus, annulus fibrosus, ligament, tendon) a large portion of the clinical reality addresses defects at the interface of two tissues. Surgical interventions that treat musculoskeletal impairments frequently reconstruct the interface between two musculosketal tissues (bone, cartilage, tendon, ligament, and muscle). For example, repair of the rotator cuff muscles entails the re-approximation of the tendinous part of one or more of the muscles into the bone, reconstruction of spinal ligaments, such as the anterior longitudinal ligament requires reattachment of the tips of the ligament into the bone, and several other examples are readily available from clinical practice. Table 1 below provides a quick summary of the basic concepts:

TABLE 1

| | Anatomical | |
|---|---|---|
| Interface Type | Example | Selected Clinical Example |
| Tendon-Bone | Tendon-bone | rotator cuff repair |
| | junction | reconstruction of tendons |
| | (defined as the | after hand and finger |
| | insertion of a | trauma |
| | muscle) | re-attachement of |
| | | tendons after neurological |
| | | disorders (quadriplegia, |
| | | cerebral palsy, a.o.) |
| Tendon-Muscle | Muscle-tendon | rotator cuff repair |
| | junction | quadriceps tendon rupture |
| | (defined in the | muscle sprains (e.g., |
| | literature as | whiplash injury) |
| | the myotendinous | |
| | junction) | |
| Muscle-Bone | Muscle-bone | tennis elbow (lateral |
| | junction | epicondylitis) |
| | (defined as the | |
| | origin of a | |
| | muscle) | |
| Ligament-Bone | Attachment of | reconstruction of the |
| | ligament into | anterior longitudinal |
| | bone | ligament |
| | | reconstruction of spinal |
| | | ligaments (supraspinous |
| | | ligt) |
| | | reconstruction of the |
| | | anterior cruciate ligament |
| | | (knee) |
| | | reconstruction of medial |
| | | collateral ligament (knee) |
| | | reconstruction of the |
| | | lateral ankle joint capsule |
| | | repair of the annulus |
| | | fibrosus after discectomy |
| | | filling the tibial and |
| | | patellar end of the central |
| | | ⅓ of the patellar tendon |
| | | after ACL reconstruction |
| Intervertebral | Attachment of | the insertion of |
| Disc-Bone | the | prosthetic devices that |
| | intervertebral | replace the function of the |
| | disc to the | intervertebral disc |
| | endplates of | the repair of the annulus |
| | the vertebral | fibrosus after surgical |
| | bodies | removal of pathological |
| | | tissues |

Based on these clinical examples there is a need to develop scaffolds that employ tissue engineering principles that focus exactly on the active remodeling and tissue regeneration at the interface of two tissues, more specifically, for example, between tendon/bone, tendon/muscle, muscle/bone, and ligament/bone. This invention is to specialize/treat the scaffolds to pre-condition it to facilitate integration at the tissue interface. For instance where the implant is to attach to bone, the tips or other desired attachment points of the scaffold can be calcified by pre-treatment with hydroxyapatite, calcium phosphates, ceramics, mineralization procedures (such as those described in Silver U.S. Pat. No. 4,970,298; Constanz U.S. Pat. No. 5,231,169; Constanz U.S. Pat. No. 5,455,231; Silver U.S. Pat. No. 5,739,286; Silver U.S. Pat. No. 5,532,217; Kwan U.S. Pat. No. 5,776,193; or Kwan U.S. Pat. No. 6,187,047), members of the BMP family, or recombinant growth factors such as rhGDF-5 to facilitate integration of the scaffold into bone and thus accelerate active new tissue formation at the tendon/bone or the ligament/bone interface, for example. Similarly, regeneration the muscle/tendon or muscle/bone interface is likely to benefit when the cellularity of the scaffolds is increased. For example, where the implant is to attach to muscle, by delivering for example bone marrow cells or stem cells from other sources (including adult-derived stem cells, post-partum-derived cells) at the muscle/tendon interface at the time of surgical reconstruction or at a later, more opportune time when active tissue remodeling is at a critical point such as during an outpatient visit during post-op follow-up.

One preferred embodiment of this invention relates to a method that combines the properties of scaffolds such as ECM, particularly SIS, with the methods of mineralizing collagen scaffolds with the goal of facilitating local bone formation there were the scaffold is attached or approximated to bone. In the embodiment where SIS is used, the functionalized or treated SIS scaffold may comprise a multi-layered device consisting of two parts: a soft tissue portion, that facilitates soft tissue remodeling and a mineralized portion, that facilitates bone in-growth.

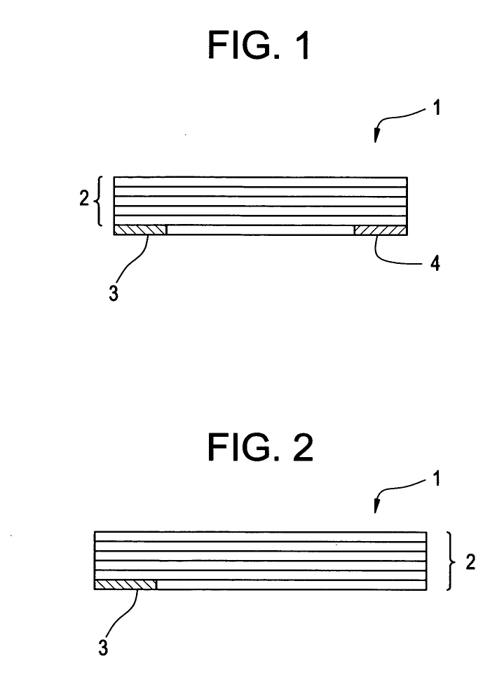

FIG. 1 depicts one embodiment of this invention, wherein device 1 comprises several layers of ECM 2 having functionalized ends 3 and 4. Ends 3 and 4 may be functionalized with the same treating composition or may be functionalized with a different treating composition. For example, if end 3 is intended to interface between device 1 and a bone, then end 3 comprises a composition that will encourage attachment between the bone and the device. If end 4 is also intended to interface with bone, then end 4 may be treated with the same, similar or different composition that that used to treat end 3. Examples of use of device 1 include, but are not limited to placement between two bones (e.g., spine (vertebrae) or appendicular skeleton (extremities) and facilitate ligament remodeling across a joint or junction between two bones (e.g., spinal ligaments, or patellar ligament, collateral ligaments at the medial or lateral aspect of the knee or elbow, the ligament complex one the lateral aspect of the ankle joint, anterior joint capsule of the glenohumeral joint (in shoulder instability, and hip joint capsule (after total hip replacement).

FIG. 2 shows another embodiment of the invention, wherein Device 1 comprises several layers of ECM 2 and single functionalized end 3. End 3 may be functionalized with a treating composition that will encourage attachment, for example, between device 1 and bone, device 1 and ligament, device 1 and muscle, and to bridge muscle-tendon-bone. For instance, in instances when whole joint does not need to be bridged by a single scaffold, the device of FIG. 2 (unlike the device of FIG. 1) can be used when the two ends of the scaffold interface with two different tissues. Clinical examples are: rotator cuff where the scaffold serves as an interface between bone and muscle, end-substance tendon tears or defects (Achilles tendon, tendon transplantations in the digits), or defects/trauma/repair of ligament-bone attachments (e.g. collateral ligaments of the ankle, knee, and elbow, or ACL, PCL).

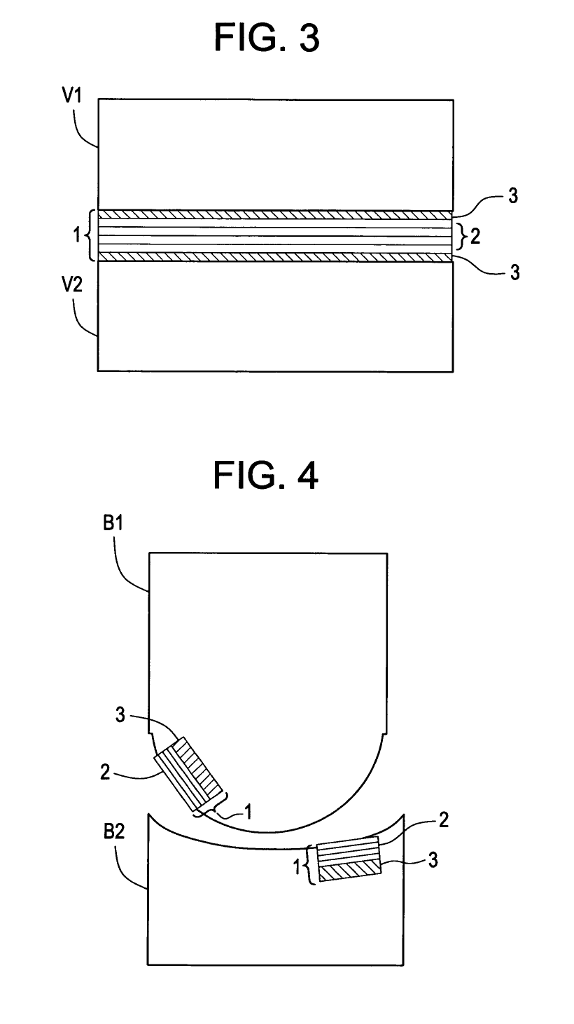

FIG. 3 represents yet another embodiment of this invention, wherein device 1 is employed in a compressive application of an intervertebral disc comprising ECM 2 and ends 3 which further comprise a treating compositions to encourage attachment of the device 1 to vertebral body V1 and vertebral body V2.

FIG. 4 represents another embodiment of this invention, wherein device 1 is used to regenerate or mimic cartilage and comprises ECM 2 and contacting end 3 treated to encourage attachment of device 1 to either bone B1 or bone B2. Bone B1 and bone B2 represent any articulating joint comprising the interface of two bones including and but not limited to joints, hips, knees, shoulders, elbows, ankles, for example.

While the above Figures have been used to describe examples that use SIS or ECM, it would be appreciated by one skilled in the art that other types of materials can be used as scaffolds in the device. Such materials useful as biocompatible scaffolds in this invention can vary, as long as they provide sufficient strength or characteristics needed to withstand the stresses required to support the intended function of the scaffold and cause little or no foreign body reaction. For example when designing to model a tendon or ligament, the tensile characteristics of the scaffold are important in order for the scaffold to successfully perform its function. In the case of compressible applications, such as in intervertebral discs or meniscus, the scaffold must have sufficient mechanical properties to withstand the physiologically required loads and flexing characteristics. In the case of cartilage repair, one skilled in the art will appreciate the continuing challenge of providing a scaffold that meets all the requirements of cartilage. In certain instances (in addition to instance that may be raised by attempting cartilage repair) such as in other avascular or limited vascular settings, it is contemplated by this invention that the scaffold may be replaced by a prosthetic device and still contain an amount of tissue interface composition material sufficient to achieve the integration of the prosthetic device with the host tissue. Where integration with host tissue is difficult, suturing or other known means of fixation may be used to complete full incorporation of the device into the host tissue. Where The scaffold can be constructed of the same or different biocompatible materials as readily determined by those of skill in the art. Sufficient strength and physical properties of the scaffold can be developed or achieved through the selection of materials used to form the device and/or from the process used to manufacture the device. In an exemplary embodiment, the device is formed from a bioresorbable or bioabsorbable material, and more preferably from a bioresorbable or bioabsorbable material that has the ability to resorb in a timely fashion in the body environment. For example, bioresorbable or bioabsorbable material can preferably resorb in less than a year. For the purposes of this invention, the terms “bioresorbable” and bioresorbable” are intended to be used interchangeably and denote a material that that is excreted from the body through normal physiological processes.

Furthermore, one skilled in the art would appreciate that the form that the scaffold can take may vary according to the desired application. For example, the scaffold may take the form of a film, foam, gel, mesh, woven or non-woven matrix.

In one embodiment of the present invention, the scaffold can be formed from a biocompatible polymer. A variety of biocompatible polymers, both bioabsorbable and nonbioabsorbable, can be used as the scaffold according to the present invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring.

In embodiments where the scaffold includes at least one synthetic polymer, suitable biocompatible synthetic polymers can include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof.

Of the foregoing, useful non-bioabsorbable polymers include, but are not limited to polyacrylates, ethylene-vinyl acetates (and other acyl-substituted cellulose acetates), polyester (Dacron®), poly(ethylene terephthalate), polypropylene, polyethylene, polyurethanes, polystyrenes, polyvinyl oxides, polyvinyl fluorides, poly(vinyl imidazoles), chlorosulphonated polyolefins, polyethylene oxides, polyvinyl alcohols (PVA), polytetrafluoroethylenes, nylons, and combinations thereof.

Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of lactide (which includes lactic acid, D,L- and meso lactide); glycolide (including glycolic acid); ε-caprolactone; p-dioxanone (1,4-dioxan-2-one); trimethylene carbonate (1,3-dioxan-2-one); alkyl derivatives of trimethylene carbonate; δ-valerolactone; β-butyrolactone; γ-butyrolactone; ε-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; α,αdiethylpropiolactone; ethylene carbonate; ethylene oxalate; 3-methyl-1,4-dioxane-2,5-dione; 3,3-diethyl-1,4-dioxan-2,5-dione; 6,6-dimethyl-dioxepan-2-one; 6,8-dioxabicycloctane-7-one and polymer blends thereof. Aliphatic polyesters used in the present invention can be homopolymers or copolymers (random, block, segmented, tapered blocks, graft, triblock, etc.) having a linear, branched or star structure. Other useful polymers include polyphosphazenes, co-, ter- and higher order mixed monomer based polymers made from L-lactide, D,L-lactide, lactic acid, glycolide, glycolic acid, para-dioxanone, trimethylene carbonate and α-caprolactone.

In one embodiment, the scaffold includes at least one natural polymer. Suitable examples of natural polymers include, but are not limited to, fibrin-based materials, collagen-based materials, hyaluronic acid-based materials, glycoprotein-based materials, cellulose-based materials, silks and combinations thereof.

In yet another embodiment, the scaffold includes a naturally occurring extracellular matrix material (“ECM”), such as that found in the stomach, bladder, alimentary, respiratory, urinary, integumentary, genital tracts, or liver basement membrane of animals. Preferably, the ECM is derived from the alimentary tract of mammals, such as cows, sheep, dogs, cats, and most preferably from the intestinal tract of pigs. The ECM is preferably small intestine submucosa (“SIS”), which can include the tunica submucosa, along with basilar portions of the tunica mucosa, particularly the lamina muscularis mucosa and the stratum compactum.

SIS has been described as a natural acellular biomaterial used to repair, support, and stabilize a wide variety of anatomical defects and traumatic injuries. See, for example, Cook® Online News Release provided by Cook Biotech Inc. at “www.cookgroup.com”. The SIS material is derived from porcine small intestinal submucosa that models the qualities of its host when implanted in human soft tissues. Further, it is taught that the SIS material provides a natural scaffold-like matrix with a three-dimensional structure and biochemical composition that attracts host cells and supports tissue remodeling. SIS products, such as OASIS and SURGISIS, are commercially available from Cook Biotech Inc., Bloomington, Ind.

Another SIS product, RESTORE Orthobiologic Implant, is available from DePuy Orthopaedics, Inc. in Warsaw, Ind. The DePuy product is described for use during rotator cuff surgery, and is provided as a resorbable framework that allows the rotator cuff tendon to regenerate. The RESTORE Implant is derived from porcine small intestine submucosa, a naturally occurring ECM (composed of mostly collagen type I (about 90% of dry weight) glycosaminoglycans and other biological molecules), which has been cleaned, disinfected, and sterilized. During seven years of preclinical testing in animals, there were no incidences of infection transmission from the implant to the host, and the RESTORE Implant has not adversely affected the systemic activity of the immune system.

While small intestine submucosa is available, other sources of ECM are known to be effective for tissue remodeling. These sources include, but are not limited to, stomach, bladder, alimentary, respiratory, or genital submucosa, or liver basement membrane. See, e.g., U.S. Pat. Nos. 6,379,710, 6,171,344, 6,099,567, and 5,554,389, hereby incorporated by reference. Further, while SIS is most often porcine derived, it is known that these various submucosa materials may be derived from non-porcine sources, including bovine and ovine sources. Additionally, the ECM material may also include partial layers of laminar muscularis mucosa, muscularis mucosa, lamina propria, stratum compactum and/or other tissue materials depending upon factors such as the source from which the ECM material was derived and the delamination procedure.

For the purposes of this invention, it is within the definition of a naturally occurring ECM to clean and/or comminute the ECM, or to cross-link the collagen within the ECM. It is also within the definition of naturally occurring extracellular matrix to fully or partially remove one or more components or subcomponents of the naturally occurring matrix. However, it is not within the definition of a naturally occurring ECM to extract, separate and purify the natural components or sub-components and reform a matrix material from purified natural components or sub-components. Also, while reference is made to SIS, it is understood that other naturally occurring ECMs (e.g., stomach, bladder, alimentary, respiratory or genital submucosa, and liver basement membrane), whatever the source (e.g., bovine, porcine, ovine) are within the scope of this invention. Thus, in this application, the terms “naturally occurring extracellular matrix” or “naturally occurring ECM” are intended to refer to extracellular matrix material that has been cleaned, disinfected, sterilized, and optionally cross-linked.

The following U.S. patents, hereby incorporated by reference, disclose the use of ECMs for the regeneration and repair of various tissues: U.S. Pat. Nos. 6,379,710; 6,187,039; 6,176,880; 6,126,686; 6,099,567; 6,096,347; 5,997,575; 5,993,844; 5,968,096; 5,955,110; 5,922,028; 5,885,619; 5,788,625; 5,762,966; 5,755,791; 5,753,267; 5,733,337; 5,711,969; 5,645,860; 5,641,518; 5,554,389; 5,516,533; 5,445,833; 5,372,821; 5,352,463; 5,281,422; and 5,275,826.

Another type of ECM is found in U.S. Pat. No. 6,042,610 to ReGen Biologics, hereby incorporated by reference, and discloses the use of a device comprising a bioabsorbable material made at least in part from purified natural fibers. The purified natural fibers are cross-linked to form the device of U.S. Pat. No. 6,042,610. The device can be used to provide augmentation for a damaged meniscus. Related U.S. Pat. Nos. 5,735,903, 5,479,033, 5,306,311, 5,007,934, and 4,880,429 also disclose a meniscal augmentation device for establishing a scaffold adapted for ingrowth of meniscal fibrochondrocytes.

In other embodiments of the present invention, the scaffold can be formed from elastomeric copolymers such as, for example, polymers having an inherent viscosity in the range of about 1.2 dL/g to 4 dL/g, more preferably about 1.2 dL/g to 2 dL/g, and most preferably about 1.4 dL/g to 2 dL/g as determined at 25° C. in a 0.1 gram per deciliter (g/dL) solution of polymer in hexafluoroisopropanol (HFIP). Suitable elastomers also preferably exhibit a high percent elongation and a low modulus, while possessing good tensile strength and good recovery characteristics. In the preferred embodiments of this invention, the elastomer exhibits a percent elongation greater than about 200 percent and preferably greater than about 500 percent. In addition to these elongation and modulus properties, the elastomers should also have a tensile strength greater than about 500 psi, preferably greater than about 1,000 psi, and a tear strength of greater than about 50 lbs/inch, preferably greater than about 80 lbs/inch.

Exemplary biocompatible elastomers include, but are not limited to, elastomeric copolymers of ε-caprolactone and glycolide with a mole ratio of ε-caprolactone to glycolide of from about 35:65 to about 65:35, more preferably from 45:55 to 35:65; elastomeric copolymers of α-caprolactone and lactide (including L-lactide, D-lactide, blends thereof, and lactic acid polymers and copolymers) where the mole ratio of ε-caprolactone to lactide is from about 95:5 to about 30:70 and more preferably from 45:55 to 30:70 or from about 95:5 to about 85:15; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40:60 to about 60:40; elastomeric copolymers of ε-caprolactone and p-dioxanone where the mole ratio of ε-caprolactone to p-dioxanone is from about from 30:70 to about 70:30; elastomeric copolymers of p-dioxanone and trimethylene carbonate where the mole ratio of p-dioxanone to trimethylene carbonate is from about 30:70 to about 70:30; elastomeric copolymers of trimethylene carbonate and glycolide (including polyglycolic acid) where the mole ratio of trimethylene carbonate to glycolide is from about 30:70 to about 70:30; elastomeric copolymers of trimethylene carbonate and lactide (including L-lactide, D-lactide, blends thereof, and lactic acid polymers and copolymers) where the mole ratio of trimethylene carbonate to lactide is from about 30:70 to about 70:30; and blends thereof. Other examples of suitable biocompatible elastomers are described in U.S. Pat. No. 5,468,253.

In another embodiment of the present invention, the scaffold can be formed from an elastomer that is a copolymer of 35:65 ε-caprolactone and glycolide, formed in a dioxane solvent and including a polydioxanone mesh. In another embodiment, the elastomer used to form the tissue repair device can be a copolymer of 40:60 ε-caprolactone and lactide with a polydioxanone mesh. In yet another embodiment, the elastomer is a 50:50 blend of a 35:65 copolymer of ε-caprolactone and glycolide and 40:60 copolymer of ε-caprolactone and lactide. The polydioxanone mesh may be in the form of a one layer thick two-dimensional mesh or a multi-layer thick three-dimensional mesh.

In yet another embodiment of the present invention, the scaffold can be formed from a polymeric foam component having pores with an open cell pore structure. The pore size can vary, but preferably, the pores are sized to allow tissue ingrowth. More preferably, the pore size is in the range of about 20 to 1000 microns, and even more preferably, in the range of about 20 to 500 microns. The polymeric foam component can, optionally, contain a reinforcing component, such as for example, the textiles disclosed above. In some embodiments where the polymeric foam component contains a reinforcing component, the foam component can be integrated with the reinforcing component such that the pores of the foam component penetrate the mesh of the reinforcing component and interlock with the reinforcing component.

It may also be desirable to use polymer blends to form scaffolds which transition from one composition to another composition in a gradient-like architecture. For example, by blending an elastomer of ε-caprolactone-co-glycolide with ε-caprolactone-co-lactide (e.g., with a mole ratio of about 5:95) a device may be formed that transitions from a softer spongy material to a stiffer more rigid material. Clearly, one skilled in the art will appreciate that other polymer blends may be used for similar gradient effects, or to provide different gradients (e.g., different absorption profiles, stress response profiles, different degrees of elasticity, or different porosities).

One of ordinary skill in the art will appreciate that the selection of a suitable material for forming the biocompatible tissue scaffold of the present invention depends on several factors. These factors include in vivo mechanical performance; cell response to the material in terms of cell attachment, proliferation, migration and differentiation; biocompatibility; and optionally, bioabsorption (or bio-degradation) kinetics. Other relevant factors include the chemical composition, spatial distribution of the constituents, the molecular weight of the polymer, and the degree of crystallinity.

The differences in the absorption time under in vivo conditions can also be the basis for combining two different copolymers when forming the device of the present invention. For example, a copolymer of 35:65 ε-caprolactone and glycolide (a relatively fast absorbing polymer) can be blended with 40:60 ε-caprolactone and L-lactide copolymer (a relatively slow absorbing polymer) to form a biocompatible scaffold. Depending upon the processing technique used, the two constituents can be either randomly inter-connected bicontinuous phases, or the constituents could have a gradient-like architecture in the form of a laminate-type composite with a well integrated interface between the two constituent layers.

The scaffold can also include a reinforcing material comprised of any absorbable or non-absorbable textile having, for example, woven, knitted, warped knitted (i.e., lace-like), non-woven, and braided structures. In one embodiment, the reinforcing material has a mesh-like structure. In any of the above structures, mechanical properties of the material can be altered by changing the density or texture of the material, the type of knit or weave of the material, the thickness of the material, or by embedding particles in the material. The mechanical properties of the material may also be altered by creating sites within the mesh where the fibers are physically bonded with each other or physically bonded with another agent, such as, for example, an adhesive or a polymer.

The fibers used to make the reinforcing component can include monofilaments, yarns, threads, braids, or bundles of fibers. These fibers can be made of any biocompatible material including bioabsorbable materials such as polylactic acid (PLA), polyglycolic acid (PGA), polycaprolactone (PCL), polydioxanone (PDO), trimethylene carbonate (TMC), copolymers or blends thereof. These fibers can also be made from any biocompatible materials based on natural polymers including silk and collagen-based materials. These fibers can also be made of any biocompatible fiber that is nonresorbable, such as, for example, polyethylene, polyethylene terephthalate, poly(tetrafluoroethylene), polycarbonate, polypropylene and poly(vinyl alcohol). In one embodiment, the fibers are formed from 95:5 copolymer of lactide and glycolide.

The scaffold, as well as any reinforcing material, may also be formed from a thin, perforation-containing elastomeric sheet with pores or perforations to allow tissue ingrowth. Such a sheet could be made of blends or copolymers of polylactic acid (PLA), polyglycolic acid (PGA), polycaprolactone (PCL), and polydioxanone (PDO).

A person skilled in the art will appreciate that one or more layers of the reinforcing material may be used to reinforce the composite implant of the invention. In addition, biodegradable textiles, such as, for example, meshes, of the same structure and chemistry or different structures and chemistries can be overlaid on top of one another to fabricate biocompatible scaffolds with superior mechanical strength.

In one embodiment, the scaffold of the present invention includes a high-density, nonwoven polymeric material. Preferably, the nonwoven material includes flexible, porous structures produced by interlocking layers or networks of fibers, filaments, or film-like filamentary structures. The polymeric material used to construct the nonwoven can include the bioabsorbable synthetic polymer materials listed above. The nonwoven may additionally include a biocompatible foam for reinforcing the scaffold.

The scaffolds of the present invention can preferably include a source of viable tissue. The source of viable tissue can vary, and the tissue source can have a variety of configurations. In one embodiment, however, the tissue is in the form of finely minced tissue fragments, which enhance the effectiveness of the regrowth and healing response. In another embodiment, the viable tissue can be in the form of a tissue slice or strip that harvested from healthy tissue that contains viable cells capable of tissue regeneration and/or remodeling. The tissue slice is preferably harvested to have a geometry that is suitable for implantation at the site of the injury or defect, and the harvested tissue slice is preferably dimensioned to allow the viable cells contained within the tissue slice to migrate out and proliferate and integrate with tissue surrounding the repair site.

Where a tissue fragment is used with the device of the present invention, the particle size of each tissue fragment can also vary. By way of non-limiting example, the tissue size can be in the range of about 0.1 and 3 mm³, in the range of about 0.5 and 1 mm³, in the range of about 1 to 2 mm³, or in the range of about 2 to 3 mm³, but preferably the tissue particle is less than 1 mm³.

Suitable tissue from which the tissue source can be derived includes, for example, cartilage tissue, meniscal tissue, ligament tissue, tendon tissue, skin tissue, bone tissue, muscle tissue, periosteal tissue, pericardial tissue, synovial tissue, nerve tissue, fat tissue, kidney tissue, bone marrow, liver tissue, bladder tissue, pancreas tissue, spleen tissue, intervertebral disc tissue, embryonic tissue, periodontal tissue, vascular tissue, blood, and combinations thereof. The tissue used to construct the tissue implant can be autogeneic tissue, allogeneic tissue, or xenogeneic tissue.

The scaffold viable tissue can also optionally be combined with a variety of other materials, including carriers, such as a gel-like carrier or an adhesive. By way of non-limiting example, the gel-like carrier can be a biological or synthetic hydrogel such as hyaluronic acid, fibrin glue, fibrin clot, collagen gel, collagen-based adhesive, alginate gel, crosslinked alginate, chitosan, synthetic acrylate-based gels, platelet rich plasma (PRP), platelet poor plasma (PPP), PRP clot, PPP clot, blood, blood clot, blood component, blood component clot, Matrigel, agarose, chitin, chitosan, polysaccharides, poly(oxyalkylene), a copolymer of poly(ethylene oxide)-poly (propylene oxide), poly(vinyl alcohol), laminin, elastin, proteoglycans, solubilized basement membrane, or combinations thereof. Suitable adhesives include, but are not limited to, hyaluronic acid, fibrin glue, fibrin clot, collagen gel, collagen-based adhesive, alginate gel, crosslinked alginate, gelatin-resorcin-formalin-based adhesive, mussel-based adhesive, dihydroxyphenylalanine (DOPA)-based adhesive, chitosan, transglutaminase, poly(amino acid)-based adhesive, cellulose-based adhesive, polysaccharide-based adhesive, synthetic acrylate-based adhesives, platelet rich plasma (PRP), platelet poor plasma (PPP), PRP clot, PPP clot, blood, blood clot, blood component, blood component clot, polyethylene glycol-based adhesive, Matrigel, Monostearoyl Glycerol co-Succinate (MGSA), Monostearoyl Glycerol co-Succinate/polyethylene glycol (MGSA/PEG) copolymers, laminin, elastin, proteoglycans, and combinations thereof.

The viable tissue can also be contacted with a specific matrix-digesting enzyme to facilitate tissue migration out of the extracellular matrix surrounding the viable tissue. The enzymes can be used to increase the rate of cell migration out of the extracellular matrix and into the tissue defect or injury, or scaffold material. Suitable matrix-digesting enzymes that can be used in the present invention include, but are not limited to the protease family including collagenases, chondroitinases, trypsin, elastase, hyaluronidasse, peptidases, thermolysin, matrix metalloproteinases, and gelatinase. Preferably, the concentration of minced tissue particles in the gel-carrier is in the range of approximately 1 to 1000 mg/cm³, and more preferably in the range of about 1 to 200 mg/cm³.

In another embodiment of the present invention, a bioactive agent may be incorporated within and/or applied to the scaffold, and/or it can be applied to the viable tissue. Preferably, the bioactive agent is incorporated within, or coated on, the device prior to the addition of viable tissue. The bioactive agent(s) can be selected from among a variety of effectors that, when present at the site of injury, promote healing and/or regeneration of the affected tissue. In addition to being compounds or agents that actually promote or expedite healing, the effectors may also include compounds or agents that prevent infection (e.g., antimicrobial agents and antibiotics), compounds or agents that reduce inflammation (e.g., anti-inflammatory agents), compounds that prevent or minimize adhesion formation, such as oxidized regenerated cellulose (e.g., INTERCEED® and SURGICEL®, available from Ethicon, Inc.), hyaluronic acid, and compounds or agents that suppress the immune system (e.g., immunosuppressants).

By way of non-limiting example, other types of effectors present within the implant of the present invention can include heterologous or autologous growth factors, proteins (including matrix proteins), peptides, antibodies, enzymes, platelets, platelet rich plasma, glycoproteins, hormones, cytokines, glycosaminoglycans, nucleic acids, analgesics, viruses, virus particles, and cell types. It is understood that one or more effectors of the same or different functionality may be incorporated within the implant.

Examples of suitable effectors include the multitude of heterologous or autologous growth factors known to promote healing and/or regeneration of injured or damaged tissue. These growth factors can be incorporated directly into the tissue repair device, or alternatively, the device can include a source of growth factors, such as for example, platelets. “Bioactive agents,” as used herein, can include one or more of the following: chemotactic agents; therapeutic agents (e.g., antibiotics, steroidal and non-steroidal analgesics and anti-inflammatories, anti-rejection agents such as immunosuppressants and anti-cancer drugs); various proteins (e.g., short term peptides, bone morphogenic proteins, glycoprotein and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGF-β I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52 aka, rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. Suitable effectors likewise include the agonists and antagonists of the agents described above. The growth factor can also include combinations of the growth factors described above. In addition, the growth factor can be autologous growth factor that is supplied by platelets in the blood. In this case, the growth factor from platelets will be an undefined cocktail of various growth factors. If other such substances have therapeutic value in the orthopaedic field, it is anticipated that at least some of these substances will have use in the present invention, and such substances should be included in the meaning of “bioactive agent” and “bioactive agents” unless expressly limited otherwise.

Biologically derived agents, suitable for use as effectors, include one or more of the following: bone (autograft, allograft, and xenograft) and derivates of bone; cartilage (autograft, allograft and xenograft), including, for example, meniscal tissue, and derivatives; ligament (autograft, allograft and xenograft) and derivatives; derivatives of intestinal tissue (autograft, allograft and xenograft), including for example submucosa; derivatives of stomach tissue (autograft, allograft and xenograft), including for example submucosa; derivatives of bladder tissue (autograft, allograft and xenograft), including for example submucosa; derivatives of alimentary tissue (autograft, allograft and xenograft), including for example submucosa; derivatives of respiratory tissue (autograft, allograft and xenograft), including for example submucosa; derivatives of genital tissue (autograft, allograft and xenograft), including for example submucosa; derivatives of liver tissue (autograft, allograft and xenograft), including for example liver basement membrane; derivatives of skin tissue; platelet rich plasma (PRP), platelet poor plasma, bone marrow aspirate, demineralized bone matrix, insulin derived growth factor, whole blood, fibrin and blood clot. Purified ECM and other collagen sources are also appropriate biologically derived agents. If other such substances have therapeutic value in the orthopaedic field, it is anticipated that at least some of these substances will have use in the present invention, and such substances should be included in the meaning of “biologically derived agent” and “biologically derived agents” unless expressly limited otherwise.

Biologically derived agents also include bioremodelable collageneous tissue matrices. The terms “bioremodelable collageneous tissue matrix” and “naturally occurring bioremodelable collageneous tissue matrix” include matrices derived from native tissue selected from the group consisting of skin, artery, vein, pericardium, heart valve, dura mater, ligament, bone, cartilage, bladder, liver, stomach, fascia and intestine, whatever the source. Although the term “naturally occurring bioremodelable collageneous tissue matrix” is intended to refer to matrix material that has been cleaned, processed, sterilized, and optionally crosslinked, it is not within the definition of a naturally occurring bioremodelable collageneous tissue matrix to purify the natural fibers and reform a matrix material from purified natural fibers.

The proteins that may be present within the scaffold include proteins that are secreted from a cell or other biological source, such as for example, a platelet, which is housed within the implant, as well as those that are present within the implant in an isolated form. The isolated form of a protein typically is one that is about 55% or greater in purity, i.e., isolated from other cellular proteins, molecules, debris, etc. More preferably, the isolated protein is one that is at least 65% pure, and most preferably one that is at least about 75 to 95% pure. Notwithstanding the above, one of ordinary skill in the art will appreciate that proteins having a purity below about 55% are still considered to be within the scope of this invention. As used herein, the term “protein” embraces glycoproteins, lipoproteins, proteoglycans, peptides, and fragments thereof. Examples of proteins useful as effectors include, but are not limited to, pleiotrophin, endothelin, tenascin, fibronectin, fibrinogen, vitronectin, V-CAM, I-CAM, N-CAM, selectin, cadherin, integrin, laminin, actin, myosin, collagen, microfilament, intermediate filament, antibody, elastin, fibrillin, and fragments thereof.

Glycosaminoglycans, highly charged polysaccharides which play a role in cellular adhesion, may also serve as effectors according to the present invention. Exemplary glycosaminoglycans useful as effectors include, but are not limited to, heparan sulfate, heparin, chondroitin sulfate, dermatan sulfate, keratan sulfate, hyaluronan (also known as hyaluronic acid), and combinations thereof.

The scaffold of the present invention can also have cells incorporated therein to serve as effectors. Suitable cell types that can serve as effectors according to this invention include, but are not limited to, osteocytes, osteoblasts, osteoclasts, fibroblasts, stem cells, pluripotent cells, chondrocyte progenitors, chondrocytes, endothelial cells, macrophages, leukocytes, adipocytes, monocytes, plasma cells, mast cells, umbilical cord cells, stromal cells, mesenchymal stem cells, epithelial cells, myoblasts, tenocytes, ligament fibroblasts, neurons, bone marrow cells, synoviocytes, embryonic stem cells; post-partum stem cells, precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells. If other cells are found to have therapeutic value in the orthopaedic field, it is anticipated that at least some of these cells will have use in the present invention, and such cells should be included within the meaning of “cell” and “cells” unless expressly limited.

Cells typically have at their surface receptor molecules which are responsive to a cognate ligand (e.g., a stimulator). A stimulator is a ligand which when in contact with its cognate receptor induce the cell possessing the receptor to produce a specific biological action. For example, in response to a stimulator (or ligand) a cell may produce significant levels of secondary messengers, like Ca⁺², which then will have subsequent effects upon cellular processes such as the phosphorylation of proteins, such as (keeping with our example) protein kinase C. In some instances, once a cell is stimulated with the proper stimulator, the cell secretes a cellular messenger usually in the form of a protein (including glycoproteins, proteoglycans, and lipoproteins). This cellular messenger can be an antibody (e.g., secreted from plasma cells), a hormone, (e.g., a paracrine, autocrine, or exocrine hormone), a cytokine, or natural or synthetic fragments thereof.

The scaffold of the invention can also be used in gene therapy techniques in which nucleic acids, viruses, or virus particles deliver a gene of interest, which encodes at least one gene product of interest, to specific cells or cell types. Accordingly, the biological effector can be a nucleic acid (e.g., DNA, RNA, or an oligonucleotide), a virus, a virus particle, or a non-viral vector. The viruses and virus particles may be, or may be derived from, DNA or RNA viruses. The gene product of interest is preferably selected from the group consisting of proteins, polypeptides, interference ribonucleic acids (iRNA) and combinations thereof.

Once the applicable nucleic acids and/or viral agents (i.e., viruses or viral particles) are incorporated into the scaffold, the scaffold can then be implanted to elicit a type of biological response. The nucleic acid or viral agent can then be taken up by the cells and any proteins that they encode can be produced locally by the cells. In one embodiment, the nucleic acid or viral agent can be taken up by the cells within the tissue fragment of minced tissue suspension, or, in an alternative embodiment, the nucleic acid or viral agent can be taken up by the cells in the tissue surrounding the site of the injured tissue. One skilled in the art will recognize that the protein produced can be a protein of the type noted above, or a similar protein that facilitates an enhanced capacity of the tissue to heal an injury or a disease, combat an infection, or reduce an inflammatory response. Nucleic acids can also be used to block the expression of unwanted gene product that may impact negatively on a tissue repair process or other normal biological processes. DNA, RNA and viral agents are often used to accomplish such an expression blocking function, which is also known as gene expression knock out.

One skilled in the art will appreciate that the identity of the bioactive agent may be determined by a surgeon, based on principles of medical science and the applicable treatment objectives. It is understood that the bioactive agent or effector of the tissue repair implant can be incorporated within the scaffold before, during, or after manufacture of the scaffold, or before, during, or after the surgical placement of the scaffold.

By way of example, a bioactive agent can be incorporated into the scaffold by placing the scaffold in a suitable container comprising the bioactive agent. After an appropriate amount time and under suitable conditions, the device will become impregnated with the bioactive agent. Alternatively, the bioactive agent can be incorporated within the scaffold by, for example, using an appropriately gauged syringe to inject the biological agent(s) into the scaffold. Other methods well known to those skilled in the art can be applied in order to load the device with an appropriate bioactive agent. Such techniques include mixing, pressing, spreading, centrifuging and placing the bioactive agent into the scaffold. Alternatively, the bioactive agent can be mixed with a gel-like carrier prior to injection into the scaffold.

In another embodiment, a surgically implanted scaffold devoid of any bioactive agent can be infused with biological agent(s), or an implant including at least one bioactive agent can be augmented with a supplemental quantity of the bioactive agent. One method of incorporating a bioactive agent within a surgically implanted device is by injection using an appropriately gauged syringe.

The amount of the bioactive agent included with a scaffold will vary depending on a variety of factors, including the size of the device, the porosity, the identity of the bioactive component, and the intended purpose of the tissue repair implant. One skilled in the art can readily determine the appropriate quantity of bioactive agent to include for a given application in order to facilitate and/or expedite the healing of tissue.

After positioning the device of the present invention within a patient, the device is preferably fastened. In one embodiment, the repair device is fixed to adjacent tissue such that the device is anchored in place. The device can be anchored to soft and/or hard tissue. In another embodiment, the device may additionally or alternatively be fixed to tissue to hold damaged or loose tissue in position. Joining the device with loose meniscal tissue provides support to the damaged tissue area and thereby facilitates rapid healing. A person skilled in the art will appreciate that a variety of techniques can be used to fix the device to hard and/or soft tissue, such as, for example, an interference fit, suture, glue, staple, tissue tack, pins, and/or other known surgical fixation techniques.

The tissue interface composition is a composition that is specialized to encourage host tissue attachment to the device of this invention. It will be apparent to one skilled in the art, that in some instances, a suitable tissue interface composition may be found in the preceding list of bioactive agents or examples of viable tissue.

In a preferred embodiment the implant of this invention comprises polarized SIS which is the result of combining two steps: 1) An initial step identical to the processing of SIS during the manufacturing of Restore® and 2) The mineralization of the SIS scaffold by using a calcium phosphate precipitation (similar to the processing of a bovine collagen scaffold such as Healos® as describe above).

With respect to increasing the cellularity of the scaffold (for instance at the muscle tendon junction), polarization of the scaffold can be obtained by delivering stem cells at the desired concentration at a specific focal point at the time of surgical repair or at a more opportune time. Preferred examples of suitable cells for this application (including cultivated cells) include but are not limited to: stem cells, bone marrow cells, fibrocytes, adipocytes, chondrocytes and combinations thereof.

More preferred examples of suitable tissue interface compositions in the instance of attaching the device of this invention to bone include, but are not limited to bioactive agents which act as osteogenic agents selected from the group consisting of hydroxyapatite, tricalcium phosphate, ceramic glass, amorphous calcium phosphate, porous ceramic particles or powders, demineralized bone particles or powder, transforming growth factors (e.g., TGF-β I-III), growth differentiation factors (e.g., GDF5, GDF6, GDF8), bone morphogenic proteins (BMP-2, BMP-4; BMP-6; BMP-7; BMP-12; BMP-13; BMP-14) 1 recombinant human growth factors (such as rhGDF-5), cartilage-derived morphogenic proteins (CDMP-1; CDMP-2, CDMP-3) and combinations thereof.

More preferred examples of suitable tissue interface compositions in the instance of attaching the device of this invention to muscle include, but are not limited to stem cells, bone marrow cells, IGF-1, HGF and combinations thereof.

More preferred examples of suitable tissue interface composition in the instance of attaching the device of this invention to fibrinocartilage, include, but are not limited to recombinant human growth factors (such as rhGDF-5), transforming growth factors (e.g., TGF-β I-III), growth differentiation factors (e.g., GDF5, GDF6, GDF8), bone morphogenic proteins (e.g., BMP-2, BMP-4, BMP-6, BMP-7, BMP-12, BMP-13, BMP-14) cartilage-derived morphogenic proteins (e.g., CDMP-1; CDMP-2, CDMP-3) and combinations thereof.

This invention also relates to a method of implanting a medical device comprising the steps of:

a) providing scaffold having at least a first end or first contact point and a second end wherein the first end or contact point further comprises a tissue interface composition that permits tissue ingrowth between the device and the host tissue; and

b) contacting the end or contact point comprising the tissue interface composition with the host tissue.

In some embodiments, the method can additionally include the step of inducing bleeding at or near the tissue defect to promote the flow of biological materials necessary for regenerating tissue or as a point of attachment of tissue.

The forgoing method can be adapted for implantation to specific host tissue, as noted by some of the following examples. Also, the foregoing description of scaffolds and tissue interface compositions are equally applicable to the methods of this invention and are hereby incorporated by reference.

Examples of when scaffolds of this invention can be attached to bone include, but are not limited to the reattachment of the rotator cuff tendon to their insertion onto the humerus, the repair and reattachment of the tendons of the digits and toes to the phalanges, the attachment of tendons after muscle translocations, the filling of the central third of the patellar tendon, reconstruction of the collateral ligaments (elbow or knee), the reconstruction of spinal ligaments, including the anterior longitudinal ligament (such as during anterior spinal procedures) or posterior ligaments (i.e. supraspinous ligament) after trauma or reconstructive surgery.

Examples of when scaffolds of this invention can be attached to muscle include, but are not limited to reattachment of the proximal part of the rotator cuff tendons to their respective muscle bellies, reattachment/augmentation of the forearm flexors (medially) or forearm extensors (laterally) after surgical debridement in epicondylitis.

Examples of when scaffolds of this invention can be attached to fibrocartilage, include, but are not limited to placing the scaffold between the endplate of a vertebra and the intervertebral disc (either the nucleus pulposus or annulus fibrosus), and the “footprint” between the humeral head and the insertion of the supraspinatus tendon.

As it would be appreciated by one skilled in the art, the foregoing disclosure and description of the present invention are illustrative and explanatory thereof and various changes in the size, shape and materials as well as in the description of the preferred embodiment may be made without departing from the spirit of the invention.

What is claimed is:

1. An implant for attaching to host tissue comprising a biocompatible scaffold having at least one end or one point for contacting the host tissue, wherein the end or point of contact further comprises a tissue interface composition that permits host tissue ingrowth between the implant and the host tissue.

2. The implant of claim 1, wherein the scaffold is bioabsorbable or nonbioabsorbable.

3. The implant of claim 1 wherein the scaffold is a synthetic or natural polymer.

4. The implant of claim 1 wherein the scaffold is selected from the group consisting of aliphatic polyesters, poly(amino acids), copy(ether-esters, polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphaenes, poly(propylene fumarate), polyurethane, poly(ester urethane), poly(ether urethane), and blends and copolymers thereof.

5. The implant of claim 1, wherein the scaffold is selected from the group consisting of collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

6. The implant of claim 1, wherein the extracellular matrix selected from the group consisting of small intestine submucosa, stomach, bladder, alimentary, respiratory, genital submucosa, liver basement membrane and combinations thereof.

7. The implant of claim 3, wherein the extracellular matrix is small intestine submucosa.

8. The implant of claim 1, wherein the scaffold is an elastomeric polymer selected from the group consisting of ε-caprolactone, glycolide, lactide, p-dioxanone, trimethylene carbonate and combinations thereof.

9. The implant of claim 1, wherein the scaffold is a polymeric foam.

10. The implant of claim 1, wherein the tissue interface composition comprises a bioactive agent.

11. The implant of claim 10, wherein the bioactive agent is selected from the group consisting of hydroxyapatite, tricalcium phosphate, ceramic glass, amorphous calcium phosphate, porous ceramic particles or powders, demineralized bone particles or powder, bone morphogenic proteins, recombinant human growth factors, cartilage derived morphogenic proteins and combinations thereof.

12. The implant of claim 11, wherein the bone morphogenic proteins are selected from the group consisting of BMP-2, BMP-4, BMP-6, BMP-7, BMP-12, BMP-13, BMP-14 and combinations thereof.

13. The implant of claim 10, wherein the bioactive agent is rhGDF-5.

14. The implant of claim 1, wherein the tissue interface composition is selected from the group consisting of stem cells, bone marrow cells, fibrocytes, adipocytes, chondrocytes and combinations thereof.

15. The implant of claim 1, wherein the tissue interface composition is selected from the group consisting of stem cells, bone marrow cells, IGF-1, HGF and combinations thereof.

16. The implant of claim 1, wherein the tissue interface composition is selected from the group consisting of recombinant human growth factors, transforming growth factors, growth differentiation factors, bone morphogenic proteins, cartilage-derived morphogenic proteins and combinations thereof.

17. The implant of claim 1, wherein the scaffold further comprises a second end or second contact point for contacting the host tissue.

18. A method of implanting a medical device comprising the steps of:
a) providing a scaffold having at least a first end or first contact point and a second end wherein the first end or contact point further comprises a tissue interface composition that permits tissue ingrowth between the device and the host tissue; and
b) contacting the end or contact point comprising the tissue interface composition with the host tissue.

19. The method of claim 21, wherein the host tissue is bone.

20. The method of claim 18, wherein the scaffold mimics a tendon.

21. The method of claim 18, wherein the scaffold mimics a ligament.

22. The method of claim 18, wherein the scaffold mimics a muscle.

23. The method of claim 18, wherein the scaffold mimics an intervertebral disc.

24. The method of claim 18, wherein the tissue is muscle.

25. The method of claim 18, wherein the scaffold is extracellular matrix.

26. The method of claim 25, wherein the extracellular matrix is SIS.

27. The method of claim 26, wherein the tissue interface composition comprises a bioactive agent.
